(19) United States
(12) Patent Application Publication
(10) Pub. No.: US 2005/0070006 A1
(43) Pub. Date: Mar. 31, 2005

(54) Method for detecting mutagenic substances

(76) Inventors: Georg Reifferscheid, Neuwied (DE); Claudia Schmid, Essen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/482,790

(22) PCT Filed: Jun. 25, 2002
(86) PCT No.: PCT/EP02/06990

(30) Foreign Application Priority Data
Jul. 4, 2001 (DE) 101 32 280.1

(51) Int. Cl. C12N 1/21

(52) U.S. Cl. 435/252.34

(57) ABSTRACT

The invention relates to a method and means for the rapid identification of mutations. The test strains used in the invention contain a reporter system and a selection marker. An antibiotic resistance protein which after mutation allows the selective growth of the revertant and the targeted induction of the inducible reporter system of the developing revertants acts as the selection marker. Proteins that are capable of directly or indirectly triggering a measuring signal are used as the reporters. The mutations are identified by means of the reporter signal of the developing revertants, in such a way that the evaluation can take place after only a few hours.

The invention relates to a method and means for the rapid detection of mutagenic substances.

Bacterial mutageneity and genotoxicity tests nowadays play a crucial role as screening methods in product development in the chemical and pharmaceutical industry and in the routine analysis of environmental samples (industrial effluent, surface waters, room air condensates, sediments, suspended matter, soil samples, etc.). The most frequently used mutation assay is the Ames (salmonella/microsome) mutageneity test, which is also part of approval procedures and toxicology tests (Ames, B. N., Lee, F. D. and Durston, W. E.; Proc. Natl. Acad. Sci. USA 70 (1973) pp. 782-786; OECD guidelines for the testing of chemicals). Other known genotoxicity tests are, for example, the umu test, whose microplate version has entered standardisation in accordance with DIN (DIN 38415-3) and ISO (Reifferscheid, G., Heil, J., Oda, Y. and Zahn, R. K., Mutation Res. 253 (1991) pp. 215-222), the SOS chromotest (Quillardet, P., Hofnung, M., Mutation Res. 147 (1985) 65-78) and the Vitotox® assay (yan-der-Lelie-D, Regniers, L., Borremans, B., Provoost, A., Verschaeve, L., Mutation Res. 389 (1997) 279-90).

The umu test, SOS chromotest and Vitotox® assay detect changes in the molecular structure of the DNA of the test organism Salmonella typhimurium or Escherichia coli caused by genotoxins. The tests are based on measurement of the induction, caused by genotoxicity, of genes of the bacterial SOS regulon. These genes are involved, for example, in a DNA repair system (SOS repair; umuDC genes in the umu test) which is susceptible to errors, in the inhibition of cell division (sfiA gene in the SOS chromo-test) and in repair and recombination (recN in the Vitotox® assay). The detection is carried out by photometric, luminometric or fluorometric measurement. The detection method depends on the type of (frequently plasmid-localised) reporter system (for example galactosidase, luciferase) which is coupled to the respective SOS gene.

The umu test is employed in State authorities concerned with environmental analysis and in companies in the chemical-pharmaceutical industry for environmental monitoring and substance testing. Although the umu test, SOS chromotest and Vitotox® assay, as indicator tests for genotoxicity, cover a wide range of primary DNA damage, they do not, however, provide direct evidence of mutations. In contrast to mutations, primary DNA damage can still be repaired correctly and is then of no significance to the affected organism.

A further genotoxicity test which is occasionally used is the Mutatox® test (Bulich-A, Schriftenr. Ver. Wasser. Boden. Lufthyg. 89 (1992) 763-70). This test method detects genotoxicity in dark mutants of the luminescent salt-water bacterium Vibrio fisheri. The precise mechanism of restoration of the luminescence after treatment with genotoxins is unexplained. However, it is assumed that reversion of the luminescence can be effected by various mutations, in particular by gene induction (in a similar way to the SOS tests umu test, SOS chromotest and Vitotox® assay) or alternatively DNA intercalation (Bulich and Ulitzur: The mode of action of genotoxic agents in the Mutatox®-Test-System, Microbics Corp., unpublished circular; Arfsten, D. P., Davenport, R., Schaeffer; D. J., Biomed. Environ. Sci. 7 (1994) 144-9). Since the test is only used occasionally, there is as yet no conclusion on the correlation to mutageneity tests such as the Ames test. In contrast to the assumption of the detection of mutations which is implied by the name ‘Mutatox®’, this test method is, like all other SOS tests, an indicator test.

In the Ames test, use is made of strains of Salmonella typhimurium which, through mutations in the histidine operon (His operon), have lost the ability to grow on histidine-free nutrient media (Agar plates with minimal nutrient medium). The measure used for the mutageneity of the sample analysed is the number of prototrophic His revertants which have formed through mutations in the His operon under the influence of genotoxic substances. The conventional Ames test is carried out with a plurality of strains of different mutagenic specificity (for example TA98, TA100, TA1535 and TA97) as a plate incorporation assay [Maron, D. M. and Ames, B. N.; Mutation Res. 113 (3-4), 1983, 173-215]. For the metabolic activation of substances which are not directly mutagenic a nd the test bacteria, the sample to be tested here is placed on the agar together with soft agar, a buffer or S9 mix. Since the test substances diffuse into the agar during the incubation time (48 hours) at different rates depending on their solubility, a precise concentration of the sample to be tested which acts on the bacteria cannot be determined. This type of application is therefore referred to as the dose (for example 100 μg/plate).

The AmesII™ test [Gee, P., Maron, D. M. and Ames, B. N.; Proc. Natl. Acad. Sci. USA 91, 1994, 11606-11610], which has been converted to a liquid culture and carried out as a fluctuation assay in 384-well microtitre plates, represents a refinement of the conventional Ames test which now enables concentration-related analyses. The growth of the His revertants causes acidification and is indicated by the colour change of a pH-dependent indicator in the selection medium. All wells which are positive with respect to the colour change are counted here without taking into account the colour intensity. Besides the frameshift mutation strain TA98, six strains (TA7001-7006), specific for the six possible base substitution types, can be employed.

In both test formats, the Ames test offers only few opportunities for automation of routine operation. The prerequisite for partial automation of the AmesII™ test is special laboratory equipment for inoculation and evaluation of 384-well microtitre plates.

Both the growth of the revertants into countable colonies (conventional Ames test) and metabolism-induced pH lowering (Amesil) require a 48-hour incubation phase, which is responsible for the long test duration. In addition, selection of the revertants via nutrient deficiency entails the risk of false positive results in the analysis of environmental mixtures which may be contaminated with nutrients. The long incubation time and the type of selection furthermore require a high degree of sterility during performance of the test in order to prevent the growth of non-test-specific bacteria or fungi.

Due to the large amount of time, work and material required, the use of the Ames test is thus limited as a screening method for large sample throughput. In addition, the position of the target gene within the chromosome makes it more difficult to analyse specifically the mutation that has taken place. The target gene for the mutation is part of a large operon consisting of a plurality of genes, which means that the actual reversion event may be masked by mutations in the other genes.

The object of the present invention was to develop a mutation test which combines the positive properties of the rapid indicator tests and the sensitive Ames test. It should be distinguished, in accordance with the known indicator tests, by rapid and automatable performance and should thus be suitable for high-throughput screening (HTP screening).

It has been found that a rapid and sensitive mutation test can be carried out with the aid of specially modified test strains from the group consisting of the Enterobacteriaceae. The strains carry a reporter system and a selection marker which is switched off by specific mutagenesis. The selection marker used is preferably an antibiotic resistance gene which enables selective growth of the revertants after back-mutation. The reporters used are proteins which are preferentially formed proportionally to the cell number. Only mutated, growing cells can express the stringently controlled reporter after induction. The measurement is carried out directly or indirectly via a colour reaction, a luminescent signal, a fluorescent signal or an electrochemical signal. This specific combination allows different mutations in the selection marker to be detected indirectly via the reporter system, which is only formed by growing cells. Direct detection of the functioning of the target gene restored by back-mutation is also possible if a selection marker having the properties of a reporter system is used. The actual mutageneity detection is not carried out via cell growth as in the prior art, but instead via expression of a reporter system, enabling evaluation after just a few hours.

The present invention therefore relates to isolated prokaryotic test strains for use in a mutageneity test which have the following features:
a) a revertible mutated selection marker in the form of a resistance gene to bacteriotoxic or bacteriostatic substances or influences,
b) a reporter system which leads directly or indirectly to a measurable signal and thus can be detected selectively in reverted bacteria.

In an embodiment, the selection marker can simultaneously also serve as reporter system. For example, the selection marker and reporter system here can be identical.

In a preferred embodiment, the selection marker is an antibiotic resistance gene to an antibiotic which has a bacteriolytic action or inhibits protein biosynthesis.

The selection marker is particularly preferably a tetracyclin resistance gene, an ampicillin resistance gene, a kanamycin resistance gene, a chloramphenicol resistance gene or a streptomycin resistance gene.

In a preferred embodiment, the reporter system, on expression, leads directly or indirectly to a colour reaction, a luminescent signal or a fluorescent signal.

In a preferred embodiment, the reporter systems used are the β-galactosidase, luciferase or β-lactamase gene or operon.

Preference is also given to test strains which additionally have one or more of the following properties:
a cell wall which is permeable to large lipophilic molecules,
a defective excision repair,
a functioning regulation unit of the SOS system
the mutator genes umuDC and/or mucAB.

The present invention also relates to a method for the detection of mutagenic agents, essentially characterised by the following steps:
a) provision of at least one test strain according to the invention;
b) incubation of the test strains from step a) with the potential mutagen;
c) selection of the revertants capable of growth;
d) if desired induction of the reporter system (superfluous if the reporter system is simultaneously the selection marker or if only detection via growth monitoring is to be carried out);
e) direct or indirect detection of the gene product of the reporter system and/or detection of the growth of the reverted strains.

In step a), the phrase “provision of at least one test strain” means that one or more test strains or a mixture of at least two test strains are provided.

In an embodiment, the selection marker in step e) serves as reporter system, meaning that detection of the gene product of the selection marker takes place as detection of the gene product of the reporter system.

In a preferred embodiment, the detection of the growth in step e) is carried out by means of a pH indicator in the medium.

The present invention also relates to a test kit for carrying out mutation tests which contains at least one test strain according to the invention.

The present invention also relates to the use of at least one test strain according to the invention for HTP screening.

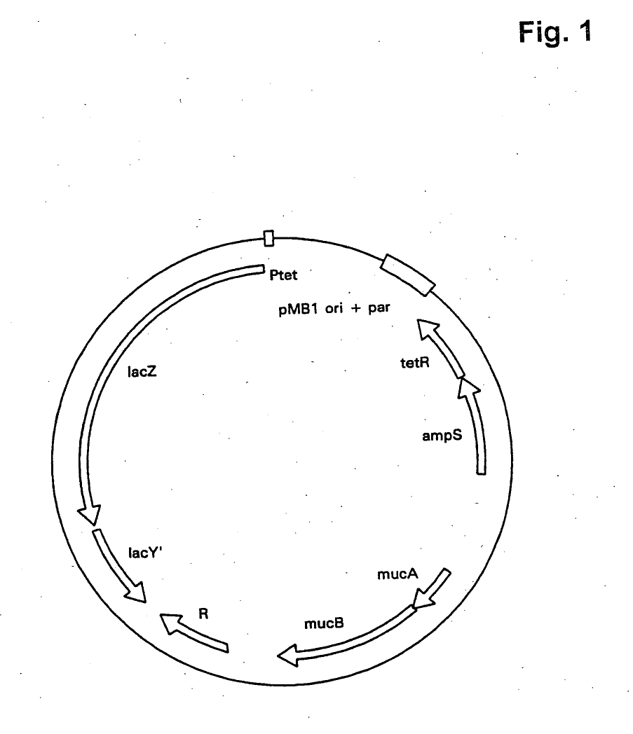

FIG. 1 shows the gene chart of the plasmid of a test strain which can be employed for the mutation test according to the invention.

Test strains which can be employed for the mutation test according to the invention contain a preferably plasmid-localised selection marker whose functioning has been switched off reversibly by specific mutagenesis, and an inducible reporter system if the selection marker is not simultaneously the reporter system.

The test strains used are bacteria. Preference is given to prokaryotic test strains from the group consisting of the Enterobacteriaceae, particularly preferably Escherichia coli or Salmonella typhimurium with a combination of specific properties determined in the genotype. The starting organism used is preferably the strains Salmonella typhimurium TA1535 (rfa, AuvrB) (McCann, J., Choi, E., Yamasaki, E. and Ames, B. E.; Proc. Natl. Acad. Sci. USA 72 (1975), pp. 5135-5139) or Escherichia coli EE122 (rfa-like mutation, ΔuvrB) (Eisenstadt, E., Warren, A. J., Potter, J., Atkins, D. and Miller, H. J.; Proc. Natl. Acad. Sci. USA 79, 1982, 1945-1949).

The strain EE122 is distinguished, for example, by robustness and an active chromosomal umuDC operon.

A multiplicity of strains which carry a selection marker and an inducible reporter system are known from the prior art. In contrast to these organisms, however, the selection marker of the test strains according to the invention has mutated revertibly with loss of function. Only the introduction of a selection marker which has mutated in this way enables a test strain to be employed for the method-according to the invention.

After treatment of the test strain with mutagenic agents, re-commencement of growth in the selection medium as a consequence of a reversion event in the target gene which encodes for the selection marker is preferably determined quantitatively by the reporter system.

The reporter system of the test strains according to the invention is preferably inducible and is preferably formed proportionally to the number of cells present. The uninduced basal level should be as low as possible. The reporter system can be an individual gene or alternatively an operon.

Suitable reporters here are gene products which can be detected directly or indirectly photometrically, fluorimetrically, luminometrically or electrochemically. Preferred gene products are GFP (green fluorescent protein), β-lactamase, luciferase and particularly preferably β-galactosidase, which can be detected in various known ways.

The advantage of GFP lies in the possibility of detection without addition of substrate. An influence on the activity of the reporter by inhibiting constituents of the test material can thus be substantially excluded.

If selection marker and reporter system are identical, direct detection of the mutation is possible. An example which may be mentioned is β-lactamase, which promotes ampicillin resistance. The activity of the ampicillinase gene restored by back-mutation can be detected directly, for example through the use of the substrate nitrocefin (O'Callaghan, C. H., Morris, A., Kirkby, S. K. and Shingler, A. H, Antimicrob. Agents Chemother. 1 (1972) 282-288; Sutton, L. D., Biedenbach, D. J., Yen, A. and Jones, R. N., Diagn. Microbiol. Infect. Dis. 21 (1995)1-8).

On the other hand, the use of β-galactosidase enables the utilisation of an amplifier effect—caused by accumulation of the product of the enzyme reaction. The alternative luminometric measurement method additionally excludes certain interfering factors, such as, for example, intensely coloured test substances.

The reporter system and/or selection marker may be present on the chromosome or on a plasmid under stringent or relaxed control with a constant copy number. The preferred number of low-copy plasmids is 15-20 copies. Uniform distribution on the daughter cells should be ensured.

The reporter system is preferably under the control of a stringently regulated, inducible promoter. Promoters of this type are known to the person skilled in the art. Use is preferably made of the tetA promoter, which is repressed by the tetR-encoded repressor protein and enables specific induction of the reporter by addition of tetracyclin or a Tet derivative, for example anhydrotetracyclin, after the manifestation of the mutation. For optimum induction of the tetA promoter in resistant (back-mutated) bacterial cells, use is preferably made of atypical tetracyclins, such as anhydrotetracyclin. The anhydrotetracyclin derivative, with an MIC (minimum inhibition concentration) value of 2 μg/ml, has lower antibiotic activity compared with 0.5 μg/ml for tetracyclin [Olivia, B., Gordon, G., McNicholas, P., Ellestad, G. and Chopra, I Antimicrob. Agents Chemother. 36, 1992, 913-919] and at the same time binds to the Tet repressor approximately 35 times more strongly [Degenkolb, J., Takahashi, M., Ellestad, G. A. and Hillen, W. Antimicrob. Agents Chemother. 35, 1991, 1591-1595].

By induction of β-galactosidase under tetA promoter control with anhydrotetracyclin, stronger signal values are achieved even at very low, non-inhibiting antibiotic concentrations.

The selection marker employed in the test strains according to the invention is a resistance gene which has been deactivated by mutation and which, after back-mutation, facilitates selective growth of the revertants. Suitable gene products of the selection markers are in general resistance proteins which facilitate targeted growth under selective conditions and whose encoding gene can be switched off reversibly by mutation. The resistance promoted by the (intact) resistance gene can be resistance to a bacteriotoxic or bacteriostatic substance, such as, for example, an antibiotic, or an effect or influence (noxa, for example temperature or radiation). The resistance gene according to the invention is preferably an antibiotic resistance gene. The selection markers used are particularly preferably resistance genes to antibiotics which inhibit protein biosynthesis or have a bacteriolytic action. This specific type of selection marker achieves not only termination of growth of the non-mutated and thus sensitive bacteria in the antibiotic-containing selection medium, but also causes the switching-off of protein biosynthesis. The signal background due to nonmutated bacteria thus remains low after induction. It must be ensured that the selection pressure is maintained over the test duration.

The selection medium used for the test strains are conventional media which, if the selection is not caused by effects such as, for example, radiation or temperature, comprise additives of the corresponding selection agent. The person skilled in the art is able to assemble suitable selection media depending on the test strain used, the particular resistance mechanism and the number of resistance genes. The concentrations of the antibiotics in the selection medium are in the usual range. In the case of ampicillin, about 50 μg/ml are preferably used. The concentrations of the antibiotics are dependent on the type of plasmid on which the resistance gene is localised, on the nature of the resistance mechanism and on the test strain used.

Due to the antibiotic resistance used in accordance with the invention for selection, fewer limits are placed on the selection of the medium than, for example, in the prior art when carrying out conventional mutageneity tests. Such tests are based, for example, on the formation of prototrophic His revertants. It is thus only possible to use minimal media in order to ensure that the selection medium comprises no histidine. For the method according to the invention, by contrast, it is possible to use any type of medium which comprises all constituents necessary for growth of the germ type used. Selection does not take place via a missing nutrient constituent, but instead preferably via the addition of an antibiotic. Full media are therefore preferably employed in accordance with the invention instead of a minimal medium. This results in better and faster growth of the reverted germs and thus facilitates particularly quick performance and evaluation of the test.

The gene serving as selection marker can, for detection of one mutation type, have a certain type of mutation. Furthermore, through differently mutated variants of the target gene on a plasmid, various mutation types can be covered using only one test strain if the spontaneous mutation rates of the combined mutation types are in the same order of magnitude. This results in a significant reduction in the amount of work and material since the use of a large number of strains and complex parallel determinations are no longer necessary.

In the production of the test strains according to the invention by targeted mutagenesis [Kunkel, T. A; Proc. Natl. Acad. Sci. USA; 82 (2) 488-92], the various mutation types (frameshift and base substitution mutations) are introduced directly into known or synthetically produced regions with particular sensitivity to mutagenic events (hot-spot regions) or into active centres of the antibiotic resistance genes by means of a mutated primer air during synthesis of the plasmid in question with the aid of the PCR polymerase chain reaction) technique and other DNA-modified enzymes. The various mutation types and suitable hot-spot regions are known to the person skilled in the art in the area of mutation tests. For example, repetitive base sequences with a high guanine content or alternating GC boxes are particularly susceptible. G-A transitions and G-T transversions occur frequently in base substitutions.

The antibiotic resistance stage is to be restored by back-mutation in the sequence section modified by deletion, insertion or exchange of bases. When modifying the sequence sections responsible for the formation of the active centre, it should be taken into account that in most cases only precise back-mutations can completely restore the functioning of the target gene. In the case of localisation of the mutation hot-spot at the beginning of the reading frame of the target gene, a longer gene region is available for back-mutation. In the case of base exchange, it is preferred either for an amino acid which is important for the functioning of the protein to be modified or alternatively for a stop codon to be introduced. This is then preferably located before the gene section which is important for the functioning of the protein. In the case of introduction of frameshift mutations, the possibility of opening alternative reading frames must be considered. Besides selection marker and reporter system, the test strains preferably have further properties which make them more sensitive and universally applicable for the test according to the invention. This is, in particular, a cell wall which is permeable to large, lipophilic molecules (for example rfa or rfa-like mutation) and/or a defective excision repair (for example mutation in the uvrA or B gene).

In addition, the test bacterium should preferably have available a functioning regulation unit of the SOS system. Various genes of the SOS system are very advantageous for sensitive and complete detection of mutagenic substances. These include the genes recA, lexA, umuDC and/or mucAB. The homologous gene-products UmuDC and MucAB are also referred to as mutator genes since they increase or firstly facilitate the formation of mutations in the bacteria during genotoxic events. Whereas umuDC is localised in functioning form on the chromosome of E. coli, the mucAB operon has been isolated from naturally occurring plasmids (McCann, J. and Ames, B. E.; Proc. Natl. Acad. Sci. USA 73 (1976) pp. 950-954). These gene products frequently act synergistically and prevent an otherwise lethal termination of replication. The gene products RecA and LexA serve for recognition of DNA damage and regulate induction of the genes of the SOS system.

Also advantageous is the lack of antibiotic resistance in the starting strain.

On use of test bacteria which do not have a chromosomal Umu operon, this or preferably the Muc operon can additionally be introduced via a plasmid. A source which can be used for the sequence of the Muc operon is the plasmid pKM101 [McCann, J., Spingarn, N. E., Kobori, J. and Ames, B. N.; Proc. Natl. Acad. Sci. USA 72(3), 1975, 979-983], which can be isolated from the strain Salmonella typhimurium TA100 or TA98. After amplification by means of PCR [Mullis, K. B. and Falaona, F. A.; Methods Enzymol. 155, 1987, 335-350] and cloning, the functioning of the Muc operon on the new plasmid can be checked in the Ames test. With the test substance nitrofurazone, which develops its mutagenic action principally via the SOS system [Bryant and McCalla; Chem. Biol. Interact. 31 (2) 1980, 151-66], a significantly increased mutation rate compared with the control should be observed in the case of the strains with the cloned Muc operon [Zeiger, E., Anderson, B., Haworth, S., Lawlor, T., Mortelmans, K. and Speck, W; Environm. Mutagen. 9 (9) 1987, 1-110].

This plasmid introduced by transformation methods must contain a replication origin which is suitable for replication in the test strain and a marker for maintenance of the plasmid during growth. In addition, the mutated target gene and the inducible reporter system can be localised on this plasmid.

The production of a plurality of plasmids, each of which only has one specific mutation, enables differentiation of the mutagens, enabling a mutation spectrum to be recorded. Mutation analysis by means of molecular-biological methods is simplified here by the preferred position of the mutated gene on the plasmid. It is furthermore possible to determine a plurality of possible mutation variants simultaneously by amplification of the mutated gene serving as selection marker with various mutation types on a plasmid.

By simultaneous use of a test strain which is analogous from the genotype point of view, but is not revertible, the cytotoxicity of the test material can be monitored.

The test strains employed hitherto (Ames test) are based on the restoration of prototrophic growth and are therefore susceptible to growth-promoting substances, which can have an adverse effect, in particular in the analysis of environmental samples having a high organic content. Selection in accordance with the invention of the revertants, preferably via antibiotic resistance, enables the generation of false positive results to be drastically reduced and contamination by non-test-specific microorganisms to be avoided.

The present invention therefore also relates to a test kit for the rapid detection of mutagenic substances. This test kit for carrying out mutation tests comprises at least one or more test strains according to the invention, which may also be present and/or employed in the form of a strain mixture. Furthermore, optional constituents, such as the substrate of the reporter system, a stop solution or specific selection or selection/indicator media may additionally be present in the test kit. These additives are typically reagents which are also commercially available independently of the test kit. In order to make it easier for users to carry out the test by the method according to the invention, the test kit preferably additionally contains test instructions and an evaluation programme. The test kits according to the invention can be used for individual detection of mutagenic substances or for HTP screening.

FIG. 1 shows the example of a plasmid of a test strain according to the invention. The test strain contains a single-copy plasmid or a low-copy plasmid having a moderate number of copies (15-20 copies). Constituents of the plasmid are:
a replication origin which guarantees a single plasmid or a moderate number of copies (for example PMBI origin)
the par region, which ensures uniform distribution of the plasmid over the daughter cells
the mutated selection gene as target gene for mutations (amps)
the constitutively expressed repressor gene for the Tet repressor protein (tetR)
the mucAB operon
a resistance gene for obtaining the plasmid (R)
the lacZ reporter system under the control of the test promoter

In order to carry out the method according to the invention, one or more test strains according to the invention are provided in a cell number of typically from 10⁷ to 10⁹/ml. Suitable media for the culture of bacteria are known to the person skilled in the art. Preference is given to the use of a full medium, such as, for example, Luria-Bertani (LB) medium.

The test can be carried out in microtitre plates or similar vessels which are suitable for bacterial culture.

Firstly, the substance to be investigated or the substance mixture to be investigated and, if desired, further reagents, such as, for example, S9 mix, are added to the cells. The cells are subsequently pre-incubated for a certain period. The selection agent is, depending on the mode of action, added immediately or alternatively at a certain time during pre-incubation. Pre-incubation is necessary, in particular, in order that the cells can come into contact for a sufficiently long period with the substances to be tested, so that a set damage is able to manifest itself as a mutation during replication. The pre-incubation is typically from 0.5 to 2 hours. The cells are subsequently incubated in the selection phase for a period of typically from 5 to 24 hours. The incubation time is dependent, inter alia, on the pre-incubation time, the incubation temperature, the selection type, on the reporter system, on the medium and the pre-dilution of the test batches. At 37° C., from 5 to 8 hours are typically required for incubation, at, for example, 28-30° C., 15-20 hours are typically required. The reporter system is subsequently induced by addition of the corresponding induction agent, and the mixture is incubated at 37° C. for typically from 1 to 2 hours. If the reporter system is identical with the selection marker, this induction step is superfluous.

The mutageneity of the substance to be investigated is determined by detection of the gene product of the reporter system. To this end, a photometric, fluorimetric, luminometric or electrochemical measurement is carried out, depending on the reporter system.

Instead of or in addition to detection of the revertants by induction of the reporter gene, these can also be detected via their growth. In particular on use of a full medium, the growth of the revertants is generally so fast that detection can take place after only about 16-18 hours. Detection of the growth can take place either via direct turbidity measurement or an indicator dye present in the medium. During the change in pH of the medium caused by the bacterial growth, a colour change of the indicator takes place. Suitable indicators should exhibit a colour change between pH 7 and pH 5. Examples of suitable indicators are, for example, Bromocresol Red or Bromothymol Blue. The indicator must not have a genotoxic potential and must not significantly affect the bacterial growth.

With the aid of this additional possibility for detection of reverted germs, two simple and sensitive detection systems are available to the user. Detection via cell growth is very simple and does not require induction of the reporter gene. Evaluation can take place by detection of the colout change. This detection method only gives qualitative results. A quantitative conclusion is possible if aliquots of the test batch are divided over a plurality of batches (for example wells) and the number of batches which exhibit a colour change is set in relation to the respective concentration of the test material. By contrast, the preferred detection via induction of the reporter gene allows a direct quantitative conclusion since the intensity of the signal can be determined.

The method according to the invention offers the advantage of being able to detect mutations very quickly. The significant shortening of the incubation time compared with previous test systems and the selection method according to the invention enable substantial automation of the test procedure. Whereas it was previously only possible to carry out 1 to 2 test cycles per week, it is now possible to carry out 4 to 5 test cycles per week.

On use of luminometric measurement methods, even just a few mutated cells can be detected within an extremely short time, so that, through the method according to the invention, a mutation test which can be carried out in a few hours is available both for substance screening in industry and for the testing of environmentally relevant substances or multicomponent mixtures.

A further advantage of the method according to the invention is the insensitivity of the test strains used to mutations in metabolically relevant genes. In mutation tests in which, owing to the selection method, minimal media have to be used, mutations in other genes relevant to metabolism and nutrient supply frequently result in significantly reduced growth of the revertants or even death of the germs. By contrast, the germs according the invention can be selected in full media, enabling the germs to take up a large number of nutrients from the medium and to react significantly less sensitively to mutations in metabolically relevant genes.

The method according to the invention is particularly suitable for use in product development in the chemical, pharmaceutical and cosmetic industries and in environmental analysis (effluent, surface waters, sediments, suspended matter, room air, pre-existing pollution, pure substances).

Even without further details, it is assumed that a person skilled in the art will be able to utilise the above description in its broadest scope. The preferred embodiments and examples should therefore merely be regarded as descriptive disclosure which is absolutely not limiting in any way.

The complete disclosure content of all applications, patents and publications listed above and below, in particular the corresponding application DE 101 32 280.1, filed on 04.07.2001, is incorporated into this application by way of reference.

### EXAMPLES

The following working example relates to the use of ampicillin-sensitive strains with a plasmid-localised mutated ampicillin resistance gene (frameshift strain and base-exchange strain) and the tetA promoter-controlled reporter system β-galactosidase. The plasmid is introduced into the test strain E. coli EE122 or Salmonella typhimurium TA1535. The detection method described is chemiluminometric measurement. The strain for detection of frameshift mutations is referred to below and in the result examples given as 4S or 6S, and the strain for detection of base-exchange mutations is referred to as 3S or 14S.

The test strains used in the working example carry a plasmid with a replication origin (pMB1) derived from pBR322 [Sutcliffe, J. G.; Cold Spring Harbor Symp. Quant. Biol. 43, 1997, 77-90]. By additional cloning of the par (‘partition’) region from pSC101 [Meacock, P. A. and Cohen, S. N., Cell 20, 1980, 529-542], stable transmission of the plasmid to the daughter cells was ensured. The donor plasmid for the second resistance gene (the cam resistance gene in the example shown) was the plasmid pBR328 (National Institute of Genetics, Yata, Japan). The mucAB operon was taken from the plasmid pGW1700 [Perry, K. L. and Walker, G. C., Nature 300, 1982, 278-281]. For stringently controlled expression of the β-galactosidase reporter gene, the lacZY gene sequence in the correct reading frame after the promoter/operator region (tet^{P/O}) of the tetA gene had to be cloned into the plasmid pASK75 [Skerra, Gene 151, 131-135, 1994]. This additionally contains the gene encoding for the tet repressor (TetR) fused transcriptionally to the constitutively expressed bla gene (ampicillin resistance), producing strong repression of the tetA promoter [Skerra, Gene 151, 131-135, 1994]. The source used for the lacZY genes was the plasmid pMC1403, in which the promoter region and first eight amino acids (including the starting ATG) of the β-galactosidase are missing [Casadaban et al., J. Bacteriol. 143, 1980, 971-980]. Since only a few restriction interfaces are available for isolation of the sequence from pMC1403, the lacZY genes were firstly cloned into the pGFPuv vector. The restriction enzymes of the multiple cloning site (MCS) now at the 5′-end of the lacZ sequence then enabled targeted cloning into the reading frame after the starting ATG of the tetA gene.

The base substitution or frameshift mutation was introduced into the lactamase gene by targeted mutagenesis. As an example of base substitution, the transition of adenine to guanine was carried out in the active centre 67 amino acids after the starting ATG. The amino acid glycine was thereby introduced instead of the amino acid serine (5′-AGC-3′→5′-GGC-3′) and consequently the β-lactamase was reversibly deactivated. As a further example of a base substitution (strain 14S), transversion of thymine to guanine was carried out 66 amino acids after the starting ATG. The amino acid arginine was thereby introduced instead of the amino acid methionine (5′-ATG-3′→5′-AGG-3′). As an example of a frameshift mutation (strain 4S), the bases guanine and cytosine were introduced in an alternating GC box nine amino acids after the active serine radical, producing a stop codon (TGA) 44 amino acids after the mutation. In a further frameshift mutation strain (strain 6S), an additional guanine was integrated 17 amino acids after the active centre, producing a stop codon 19 amino acids after the mutation. In all cases, the β-lactamase was reversibly deactivated.

The plasmid was introduced into the strain Escherichia coli EE122 [Δ(lac proB) Δ(uvrB gal bio) ara thi rfa] [Eisenstadt, E., Warren, A. J., Porter, J., Atkins, D. and Miller, J. H., Proc. Natl. Acad. Sci. USA 79, 1982, 1945-1949].

1. Pre-Culture

The bacteria (3S, 14S, 4S and 6S) are incubated overnight (or for at least 3 to 4 hours) in an incubator with shaking at 37° C. in Luria-Bertani (LB) medium with addition of the antibiotic in order to maintain the plasmid. Next morning (or after 3 to 4 hours), the bacteria are adjusted to a cell number of about 2.5×10⁷ (corresponds to E₆₀₀ of about 0.05) using LB medium and kept on ice until incubation with the test substances.

2. Preparation of the Test Plates and Incubation

2.1 Pre-Incubation Phase

The pre-incubation can be carried out, for example, using 24-well or 96-well test plates.

During incubation without S9 mix, 10 μl of various concentrations of the test substance, the negative-control substance (solvent control) and the positive-control substance in amounts of 1 ml in each case are added to the bacteria adjusted in accordance with section 1. In the case of substances which require S9 in order to develop their mutagenic potential, an S9 cofactor mix and S9 are added. The cofactor mix is always prepared freshly on the test day by the method of Maron and Ames (Maron and Ames; Mutation Res. 113, 1983, 173-215). For incubation with S9, 1 ml of the bacteria adjusted in accordance with section 1 is initially introduced. 70 μl of cofactor mix and 10 μl of S9 are added. 10 μl of the substances to be tested are then added. The batch is mixed well.

This is followed by pre-incubation at 37° C. for 90 minutes with shaking.

2.2 Selection Phase

After the pre-incubation, the test batches are diluted 1:10 with LB medium (+50 μg/ml of ampicillin) and incubated for from 6 (short-term test at 37° C.) to 16 hours (long-term test at 28-30° C.) in 24-well, 96-well or 384-well plates.

3. Induction of the Reporter System

For induction of the reporter system, 100-200 ng/ml of anhydrotetracyclin are added to the batches, which are then incubated at 37° C. for 1-2 hours.

4. Measurement of the Mutageneity

For the luminometric enzyme test of the reporter system, 60 μl of the bacterial suspension are mixed with 90 μl of lyse buffer and 30 μl of substrate solution (Galacton-Plus®; Applied Biosystems, PE Deutschland GmbH). In order to terminate the reaction after incubation at 28° C. for 30 minutes, 120 μl of stop solution are added. 100 μl of the total batch are subsequently removed for the luminometric measurement. After automatic injection of 100 μl of luminescence enhancer solution (luminescent enhancer, Emerald@; Applied Biosystems, PE Deutschland GmbH), each batch is measured for a period of 5 seconds in the luminometer after a waiting time of 2 seconds.

For measurement of the chemiluminescence, 96-well luminometers (for example Labsystems, Luminoscan) or single-channel luminometers (for example Berthold, Lumat) can be used.

5. Toxicity Control

The batch for toxicity control is carried out analogously to the batch for mutageneity testing. The test strain that can be used is a strain which is non-revertible, but is otherwise identical from the point of view of the remaining genotype.

The toxicity control can likewise be carried out with the revertible test strains. The batch for this purpose is analogous to the batch for mutageneity testing. After pre-incubation for 90 minutes without selection agent, the optical density of the test batches (E₆₀₀ nm) is measured immediately.

If the measured E₆₀₀ values of the substance batches are lower than those of the control batches, cytotoxicity is present.

Indications of the toxicity of a mutagenic substance can also be drawn from the shape of the curve of the rlu values. A test substance can be taken to have an increasingly toxic action as soon as the curve of the rlu values begins to flatten out and possibly drops with further increasing substance concentrations.

6. Evaluation

The raw data (rlu values: relative luminescence units) are transferred into an MS Excel evaluation program. The rlu values minus the blank control and the ratios of the test batches against control batches are calculated. In the example presented here, a substance is regarded as mutagenic if the signal/background ratio reaches the factor 2 at at least 2 concentrations and a concentration/effect relationship exists.

### Result Example 1

Short-term test: pre-incubation: 90 min.; selection phase: 6 hours

| | |
|---|---|
| Strain | 3S (base substitution) |
| Substance | N-Methyl-N-nitro-N-nitrosoguanidine (MNNG) |

| Concentration | Relative luminescence | Mutation induction |
|---|---|---|
| [μg/ml] | units (rlu) | (factor) |
| 0 | 5446 | 1.0 |
| 0.625 | 5766 | 1.1 |
| 1.25 | 5869 | 1.1 |
| 2.5 | 12564 | 2.3 |
| 5 | 57071 | 10 |
| 10 | 159775 | 29 |

| | |
|---|---|
| Strain | 3S (base substitution) |
| Substance | Ethyl methanesulfonate (EMS) |

| Concentration | Relative luminescence | Mutation induction |
|---|---|---|
| [μg/ml] | units (rlu) | (factor) |
| 0 | 4255 | 1.0 |
| 750 | 4515 | 1.1 |
| 1500 | 17541 | 4.1 |
| 3000 | 40879 | 9.6 |
| 6000 | 16047 | 3.8 |
| 12000 | 15112 | 3.6 |

Long-term test: pre-incubation: 90 min.; selection phase: 16 hours

| | |
|---|---|
| Strain | 3S (base substitution) |
| Substance | N-Methyl-N-nitro-N-nitrosoguanidine |
| | (MNNG) |

| Concentration | Relative luminescence | Mutation induction |
|---|---|---|
| [μg/ml] | units (rlu) | (factor) |
| 0 | 4909 | 1 |
| 0.125 | 14369 | 2.9 |
| 0.25 | 104568 | 21.3 |
| 0.5 | 181601 | 37 |
| 1 | 395235 | 80.5 |
| 2 | 516442 | 105 |

| | |
|---|---|
| Strain | 4S (frameshift mutation) |
| Substance | Nitropyrene (NP) |

| Concentration | Relative luminescence | Mutation induction |
|---|---|---|
| [μg/ml] | units (rlu) | (factor) |
| 0 | 5363 | 1 |
| 0.031 | 4125 | 0.8 |
| 0.0625 | 105949 | 19.8 |
| 0.125 | 219636 | 41 |
| 0.25 | 348410 | 65 |
| 0.5 | 950853 | 177 |
| 1 | 552390 | 103 |

| | |
|---|---|
| Strain | 4S (frameshift mutation) |
| Substance | Nitrofluorene (NFL) |

| Concentration | Relative luminescence | Mutation induction |
|---|---|---|
| [μg/ml] | units (rlu) | (factor) |
| 0 | 5339 | 1 |
| 0.031 | 210703 | 39 |
| 0.0625 | 480532 | 90 |
| 0.125 | 900022 | 169 |
| 0.25 | 1713083 | 321 |
| 0.5 | 3643737 | 682 |
| 1 | 3360982 | 630 |

| | |
|---|---|
| Strain | 6S (frameshift mutation) |
| Substance | 2-Methoxy-6-chloro-9-(3-(2-chloro- |
| | ethyl)aminopropylamino)acridine, dihydrochloride |
| | (ICR-191) |

| Concentration | Relative luminescence | Mutation induction |
|---|---|---|
| [μg/ml] | units (rlu) | (factor) |
| 0 | 5369 | 1 |
| 0.016 | 151340 | 28 |
| 0.031 | 307828 | 57 |
| 0.0625 | 466003 | 86 |
| 0.125 | 685471 | 127 |
| 0.25 | 880208 | 162 |
| 0.5 | 1274327 | 235 |
| 1 | 1432620 | 264 |

### Result Example 2

Long-Term Test and Detection Via Colour Change (Bromocresol Red)

If the long-term test is carried out, the detection of the revertants can also take place via a colour change (through addition of, for example, Bromo-cresol Red to the selection medium). After pre-incubation, the bacteria are diluted with LB medium (+ampicillin+Bromocresol Red) and incubated at 37° C. for 16-18 hours in 24-well, 96-well or 384-well plates. Induction of the reporter gene is superfluous. The evaluation is carried out either by count-ing the ‘positive’ wells (the yellow wells in the case of Bromocresol Red) or by measurement using a photometer.

| | |
|---|---|
| Strain | 3S (base substitution) |
| Substance | N-Methyl-N-nitro-N-nitrosoguanidine |
| | (MNNG) |

| Concentration | | Mutation induction |
|---|---|---|
| [μg/ml] | % of positive wells | (factor) |
| 0 | 2.8 | 1 |
| 0.12 | 12.5 | 4.5 |
| 0.25 | 69 | 24.6 |
| 0.5 | 75 | 26.8 |
| 1.2 | 93 | 33.2 |
| 2.4 | 100 | 35.7 |
| 4.8 | 99 | 35.4 |
| 9.6 | 15.3* | 5.5 |
| 19.2 | 2.1* | 0.75 |

*Drop in the number of positive wells due to cytotoxicity at high
MNNG concentrations

| | |
|---|---|
| Strain | 4S (frameshift mutation) |
| Substance | 2-Methoxy-6-chloro-9-(3-(2-chloro- |
| | ethyl)aminopropylamino)acridine, dihydrochloride |
| | (ICR-191) |

| Concentration | | Mutation induction |
|---|---|---|
| [μg/ml] | % of positive wells | (factor) |
| 0 | 3.1 | 1 |
| 0.03 | 0 | 0 |
| 0.06 | 6.3 | 2 |
| 0.12 | 12.5 | 4 |
| 0.25 | 25 | 8.1 |
| 0.5 | 37.5 | 12.1 |
| 1 | 75 | 24.2 |

| | |
|---|---|
| Strain | 4S (frameshift mutation) |
| Substance | 2-Aminofluorene (+S9 mix) |

| Concentration | | Mutation induction |
|---|---|---|
| [μg/ml] | % of positive wells | (factor) |
| 0 | 16 | 1 |
| 0.031 | 13.9 | 0.9 |
| 0.063 | 10.3 | 0.6 |
| 0.13 | 57.7 | 3.6 |
| 0.25 | 81.3 | 5.1 |
| 0.5 | 100 | 6.3 |
| 1 | 100 | 6.3 |
| 2 | 100 | 6.3 |

| | |
|---|---|
| Strain | 6S (frameshift mutation) |
| Substance | 4-NQO |

| Concentration | | Mutation induction |
|---|---|---|
| [μg/ml] | % of positive wells | (factor) |
| 0 | 11 | 1 |
| 0.008 | 13.1 | 1.2 |
| 0.015 | 34.8 | 3.2 |
| 0.031 | 50.6 | 4.6 |
| 0.063 | 77.7 | 7.1 |
| 0.125 | 87.5 | 8.0 |
| 0.25 | 52* | 4.7 |
| 0.5 | 2.7* | 0.2 |

*Drop in the number of positive wells due to cytotoxicity at high
NQO concentrations

| | |
|---|---|
| Strain | 14S (frameshift mutation) |
| Substance | 4-NQO |

| Concentration | | Mutation induction |
|---|---|---|
| [μg/ml] | % of positive wells | (factor) |
| 0 | 6.9 | 1 |
| 0.015 | 22.3 | 3.2 |
| 0.031 | 36.8 | 5.3 |
| 0.063 | 72.3 | 10.5 |
| 0.125 | 89 | 12.9 |
| 0.25 | 81.3* | 11.8 |
| 0.5 | 30.6* | 4.4 |
| 1 | 2.1* | 0.3 |

*Drop in the number of positive wells due to cytotoxicity at high
NQO concentrations

### Result Example 3

Long-term test and detection via colour change (β-lactamase activity)

If the long-term test is carried out, the revertants can also be detected by direct detection of the β-lactamase activity restored after back-mutation. After pre-incubation, the bacteria are diluted with LB medium (+ampicillin) and incubated at 37° C. for 16-18 hours in 24-well, 96-well or 384-well plates. Induction of the reporter gene is superfluous. After addition of 50 μl of β-lactamase substrate (for example nitrocefin [test concentration 50 μg/ml]), the test batches are incubated for from 5 to 10 minutes.

The evaluation is carried out either by counting the ‘positive’ wells or by measurement using a photometer (nitrocefin: E492 nm).

| Strain | 3Smuc (base substitution) | |
|---|---|---|
| Substance | N-Methyl-N-nitro-N-nitrosoguanidine (MNNG) | |
| Concentration | | Mutation induction |
| [μg/ml] | % of positive wells | (factor) |
| 0 | 1 | 1 |
| 0.03 | 1 | 1 |
| 0.06 | 1 | 1 |
| 0.13 | 22 | 22 |
| 0.25 | 48 | 48 |
| 0.5 | 48 | 48 |
| 1 | 48 | 48 |
| 2 | 47 | 47 |

1. Isolated prokaryotic test strain for the detection of mutagens, at least comprising
a) a revertible mutated selection marker in the form of a resistance gene to bacteriotoxic or bacteriostatic substances or influences;
b) a reporter system whose expression can be detected selectively in reverted bacteria.

2. Test strain according to claim 1, characterised in that the selection marker simultaneously also serves as reporter system.

3. Test strain according to claim 1, characterised in that the selection marker is an antibiotic resistance gene to an antibiotic which has a bacteriolytic action or inhibits protein biosynthesis.

4. Test strain according to claim 1 one or more of claims 1 to 3, characterised in that the selection marker is a tetracyclin resistance gene, an ampicillin resistance gene, a kanamycin resistance gene, a chloramphenicol resistance gene or a streptomycin resistance gene.

5. Test strain according to claim 1, characterised in that the reporter system, on expression, leads directly or indirectly to a colour reaction, a luminescent signal or a fluorescent signal.

6. Test strain according to claim 1, characterised in that the reporter system used is the β-galactosidase, β-lactamase or luciferase gene or operon.

7. Test strain according to claim 1, additionally having one or more of the following properties:
a) a cell wall which is permeable to large, lipophilic molecules;
b) a defective excision repair;
c) a functioning regulation unit of the SOS system;
d) the mutator genes umuDC and/or mucAB.

8. Method for the detection of mutagens, essentially characterised by the following steps:
a) provision of at least one test strain corresponding to claim 1;
b) incubation of the test strain with the potential mutagen;
c) selection of the revertants;
d) if desired induction of the reporter system;
e) detection of the gene product of the reporter system and/or detection of the growth of the reverted strains.

9. Method according to claim 8, characterised in that in step e), the detection of the gene product of the selection marker takes place as detection of the gene product of the reporter system.

10. Method according to claim 8 or 9, characterised in that the detection of the growth in step e) is carried out by means of a pH indicator in the medium.

11. Test kit for carrying out mutation tests, essentially containing one or more test strains corresponding to claim 1.

12. Use of at least one test strain according to claim 1 for HTP screening.
